(19) United States
(12) Patent Application Publication
(10) Pub. No.: US 2022/0096684 A1
(43) Pub. Date: Mar. 31, 2022

(54) VEHICLE HAVING DISINFECTION SYSTEM

(71) Applicant: Saf-T-Glo Limited, Cwmbran (GB)

(72) Inventors: Robert Trundle, Cwmbran (GB); Pierre Michard, Cwmbran (GB); Mark Lipyeat, Cwmbran (GB)

(73) Assignee: Saf-T-Glo Limited, Cwmbran (GB)

(21) Appl. No.: 17/489,523

(22) Filed: Sep. 29, 2021

(30) Foreign Application Priority Data
Sep. 30, 2020 (GB) 2015526.3

(51) Int. Cl. A61L 2/10; A61L 2/24; A61L 9/20

(52) U.S. Cl. CPC A61L 2/10; A61L 2/24; A61L 9/20; B64F 5/30; A61L 2202/14; A61L 2202/15; A61L 2202/25; A61L 2202/11

(57) ABSTRACT

A vehicle includes a disinfection system for combating biological, viral, fungal and parasitic agents. The vehicle includes a power source and is configured to generate at least one signal indicative the vehicle operation status. The disinfection system includes at least one electromagnetic emission device arranged to emit electromagnetic radiation; a controller having a plurality of stored scenarios. The controller is configured to receive the signal and to use the signal to select a scenario from the plurality of stored scenarios. The controller has output means to output control signals to the at least one electromagnetic emission device to control at least one of the intensity, wavelength, and duration of the emitted electromagnetic radiation in response to the selected scenario.

### CROSS-REFERENCE TO RELATED PATENT APPLICATIONS

The present application claims priority to and the benefit of United Kingdom Priority Application No. 2015526.3, filed Sep. 30, 2020, the disclosure of which, including the specification, drawings, claims and abstract, is incorporated herein by reference in its entirety.

### BACKGROUND

This application is concerned with disinfection of vehicles and systems for carrying out disinfection of vehicles and specifically the disinfection of aircraft.

It is known that ultraviolet (UV) emissions can be used to disinfect air and/or surfaces. Such emissions have been used in medical situations. It has been widely recognized that the UVC spectrum (200 nm to 280 nm) may be used to inactivate airborne pathogens. UVC emissions may also be used to inactivate pathogens on surfaces.

U.S. Pat. No. 10,532,122 discloses a mobile air purification device in the form of an air-surface disinfector which comprises a tubular member having UV light sources arranged to irradiate an internal area and also having UV light sources arranged to irradiate an area around the device.

CN201182765 describes a UV sterilizing lamp comprises a lamp tube arranged to emit at 253.7 nm and 185 nm. The two bands are arranged to simultaneously sterilize with the 253.7 nm band and purification with the 185 nm wavelengths. It is suggested that the light can be issued in air-conditioning in trains or vehicles or in water treatments facilities.

US20060017025 discloses a portable high-intensity ultraviolet (UV-C) light gun which is arranged to emit a focused beam of UVC light that can be directed to a target.

It is an object of the invention to provide improved, flexible systems of disinfection and sterilization of vehicles and specifically disinfection of aircraft.

### SUMMARY

An exemplary embodiment relates to a vehicle having a disinfection system arranged to combat biological, viral, fungal and parasitic agents. The vehicle has a power source, an operational status, and being arranged to generate at least one vehicle output indicative of the vehicle operational status. The disinfection system includes at least one electromagnetic emission device arranged to emit electromagnetic radiation having at least one of a specified intensity, a specified wavelength, and a specified duration. A controller has a processor means and a memory means, and a plurality of scenarios are arranged to be stored in the memory means. The controller is arranged to receive a signal indicative of operational status. The controller is arranged to use the signal indicative of the operational status to select a scenario from the plurality of scenarios stored in the memory means. The controller has output means arranged to output first output control signals in response to the selected scenario, to the at least one electromagnetic emission device to control at least one of the intensity, wavelength, and or duration of the emitted electromagnetic radiation. The at least one electromagnetic emission device emits radiation in response to the first output control signals.

Another exemplary embodiment relates to a disinfection system for a vehicle, the disinfection system having a controller having a memory means and at least one electromagnetic emission device. The or each electromagnetic emission device is arranged to emit radiation having a specified characteristic, the specified characteristic being selected from the group consisting of wavelength, intensity, and duration. The controller is arranged receive signals from a vehicle in which the system is installed in use, the signals being indicative of an operational status of the vehicle. The controller is arranged to select a scenario from a plurality of scenarios stored in the memory means in response to the signal indicative of the operational status and the controller being arranged to output signals to control the specified characteristic emitted by the or each electromagnetic emission device in response to the selected scenario.

Another exemplary embodiment relates to a vehicle having at least one surface to reflect at least 80% UV radiation.

### BRIEF DESCRIPTION OF THE DRAWINGS

The invention will now be further described with reference to the following figures in which:

FIG. 1 is schematic view of an aircraft in accordance with the invention;

FIG. 2 is a schematic view of a ground vehicle in accordance with the invention

FIG. 3 is a schematic view of a ship in accordance with the invention

FIG. 4 is schematic indication of a top level architecture of the system;

FIG. 5 is a schematic of the system;

FIG. 6 is a schematic cross section of an electromagnetic emission device; and

FIG. 7 is a top view of the electromagnetic emission device of FIG. 6.

### DETAILED DESCRIPTION

According to an aspect of the invention there is provided a vehicle having a disinfection system arranged to combat biological viral, fungal and parasitic agents,

the vehicle having a power source,

the vehicle further being arranged to generate at least one vehicle output; and

the disinfection system comprising:
at least one electromagnetic emission device arranged to emit electromagnetic radiation having at least one of:
   a specified intensity;
   a specified wavelength;
   a specified dosage;
   a specified duration;

a controller having a processor means and a memory means;
the controller having at least one scenario stored in the memory means;
the controller having output means arranged to output control signals to the at least one electromagnetic emission device to control at least one of the intensity, wavelength, dosage and duration of the emitted electromagnetic radiation;

the controller being arranged:
to receive the at least one vehicle output,
to select a first scenario from the at least one stored scenario in response to the at least one vehicle output; and
to generate first output signals in accordance with the selected first scenario;

the controller having an output means arranged to output the first output control signals to the at least one electromagnetic emission device and wherein the at least one electromagnetic emission device emits radiation in response to the first output control signals.

According to a first aspect of the invention there is provided a vehicle having a disinfection system arranged to combat biological, viral, fungal and parasitic agents,

the vehicle having a power source,

the vehicle further being arranged to generate at least one vehicle output indicative of the vehicle operation status; and

the disinfection system comprising:
at least one electromagnetic emission device arranged to emit electromagnetic radiation having at least one of:
   a specified intensity;
   a specified wavelength;
   a specified duration;

a controller having a processor means and a memory means;
the controller having a plurality of scenarios stored in the memory means;
the controller being arranged to receive the signal indicative of operational status and the controller being further arranged to use the signal indicative of the operational status to select a scenario from the plurality of scenarios stored in the memory means;
the controller having output means arranged to output control signals to the at least one electromagnetic emission device to control at least one of the intensity, wavelength and duration of the emitted electromagnetic radiation in response to the selected scenario;

the controller having an output means arranged to output the first output control signals to the at least one electromagnetic emission device and wherein the at least one electromagnetic emission device emits radiation in response to the first output control signals.

The or each scenario may be provided in the form of a data table. The scenario may contain information indicative of the vehicle. The scenario may vary with the type of vehicle. The scenario may contain output information arranged to generate output signals controlling the intensity, duration, wavelength or wavelengths of electromagnetic radiation emitted.

The controller is provided with a number of stored scenarios. In some embodiments the controller may have two, three, four, five or more stored scenarios. The scenarios may contain details of the vehicle status, the operational status or details, occupancy, threat type, threat details.

The or each scenario may contain details of the vehicle status such as type of vehicle, usage of the vehicle, configuration of the vehicle, typical occupancy or potential occupancy of the vehicle.

Each scenario may be applicable to particular situation. For example an aircraft may be provided with a scenario operational status such as for embarkation, for taxiing, take-off, climbing, cruising, descent; approach and landing and disembarkation. Similarly a military vehicle may have a scenario for operational status such as maintenance; for peace-time maneuvers; for alert; for combat etc. A submarine mission usage may for example provide scenarios operational status such as for recovering objects; systems manipulation/implantation or control; disabling or removing objects; gatekeeper; diver or special operations support; protection of national assets on the seabed; forensics or investigations.

Each scenario may contain an occupancy indicator. Each scenario may be adapted to provide output signals to control electromagnetic emissions dependent on the occupancy of the vehicle.

The disinfection system may be arranged to output disinfecting electromagnetic radiation in more than one scenario. The disinfection system may be arranged to output electromagnetic radiation at more than one wavelength.

Each scenario may contain details of a threat to be counteracted. The threat may comprise a biological threat. In some embodiments the threat may comprise details of a bacteria, a fungus, a parasite or a virus. The scenario may control output signals to control electromagnetic emissions dependent on the details of the threat to the vehicle.

In some embodiments the system may be arranged to be multi-spectrum. The system may comprise emission devices arranged to emit electromagnetic radiation in more than one portion of the spectrum. The electromagnetic radiation emitted may comprise more than one UV wavelength. The electromagnetic radiation emitted may comprise more than one range of UV wavelengths. In some embodiments the electromagnetic radiation emitted may comprise UV wavelengths and IR wavelengths. The system may comprise emission devices arranged to emit electromagnetic radiation in more than one portion of the electromagnetic spectrum. In other embodiments the system may comprise a first number of emission devices arranged to emit electromagnetic radiation in a first portion of the spectrum and may comprise a second number of further devices arranged to emit electromagnetic radiation in a second portion of the spectrum.

The scenario or scenarios are stored in the memory means of the controller. The memory means may comprise a SSD or other storage means.

In some embodiments the memory means is integral with the controller. In other embodiments the memory means may be provided separately from the controller but in communication with the controller.

The controller is arranged to receive the at least one output from the vehicle.

In some embodiments the vehicle may output a number of signals. The signals may be indicative of a status of the vehicle. The status may for example be whether the vehicle is stationary, moving, location, occupied or unoccupied, combat management system of the vehicle, door status (open, shut, locked), external input such as a biological threat, boost, or an override. The status may be an output from a vehicle mission usage signal from the vehicle.

The scenario may comprise data relating to at least one of thermal requirements of the vehicle; environmental requirements of the vehicle; or electromagnetic interference (EMI) requirements of the vehicle. In some embodiments the scenario may comprise requirements for the particular vehicle. The requirements may comprise relevant sections of, for example, one or more of:

Radio Technical Commission for Aeronautics Document (RTCA DO-160G);

Software Considerations in Airborne Systems and Equipment Certification (DO-178C),

Environmental Conditions and Test Procedures for Airborne Equipment (DO-160),

UK Defense Standardization Defense Standards;

United States Military Standard—(MIL-STD);

NATO standards ALLIED ENVIRONMENTAL CONDITIONS AND TEST PUBLICATIONS (AECTP)

The scenario may in some cases comprises at least in part a mission profile data table. The scenario may in some embodiments comprise data relating to vehicle. The mission profile data table may be provided as a part of the specification of the vehicle. In some embodiments the mission profile data table may comprise data that can be updated. The data that can be updated may comprise data and or information relative to a newly identified threat. The threat may comprise a biological threat. In some embodiments the threat may comprise details of a bacteria, a fungus, a parasite or a virus.

Electromagnetic interference is defined as unwanted electrical signals and may comprise a conducted or radiated emission signal. The EMI signature may be regarded as an electromagnetic footprint. Certain vehicles may have specified EMI requirements and it may be a safety requirement that EMI signature is attenuated (that is generally limited). In some vehicles it may be a requirement that the EMI footprint is controlled in specific wavelengths or in certain portions of the vehicle.

In some embodiments the vehicle output to the controller may comprise data from movement detection sensors; proximity sensors or switches; status inputs; aircraft weight on wheel sensors; location coordinates; combat management system data/status; thermal sensors, occupancy detectors, door status locks or vehicle mission usage. It will be appreciated that the skilled person will be able to suggest a number of vehicle outputs other than those examples illustrated above.

In a preferred embodiment the disinfection system comprises a plurality of electromagnetic emission devices arranged to emit electromagnetic radiation.

In some embodiments the disinfection system comprises a plurality of electromagnetic emission devices emitting at the same wavelength.

In some embodiments the disinfection system comprises a plurality of electromagnetic emission devices emitting electromagnetic emission devices at least at a first wavelength and a second wavelength.

The electromagnetic radiation may be in the range from 1 nm to 2000 nm or more preferably from 10 nm to 800 nm. In a preferred embodiment the electromagnetic emission devices are preferably arranged to emit electromagnetic radiation in the range of UV (ultraviolet) to IR (infrared). It will be appreciated that the person skilled in the art is able to use other ranges of electromagnetic radiation depending on the desired application.

In some preferred embodiments the electromagnetic radiation may be UV light. UV light is considered to be electromagnetic radiation in the range from 10 nm to 400 nm. In some embodiments the range of emissions may be from 100 nm to 280 nm, that, is UV-C emissions. In other embodiments the emissions may be from 280 nm to 315 nm, that is, UVB emissions. In other embodiments the emissions may be from 315 nm to 400 nm, that is, UVA emissions. It may be that other ranges of emissions may be used such as extreme ultraviolet which is from 10 nm to 122 nm. It has been found that emissions in the range from 200 nm to 280 nm are particularly effective against biological pathogens. The emissions are particularly effective and cause rapid and significant DNA and RNA damage.

In some embodiments the electromagnetic emission devices may be arranged to emit radiation in more than one range or at more than one wavelength. It may for example be desirable to emit radiation having a peak wavelength substantially about 185 nm, as a first portion of the spectrum. It will for example be appreciated that emissions from 250 nm to 260 nm are particularly effective against some pathogens. It may for example be desirable to emit radiation having a peak wavelength substantially about 254 nm or 253.7 nm. Emission devices may be arranged to emit radiation substantially about 254 nm as a second portion of the spectrum. The emission devices maybe arranged to emit wavelengths in the first and the second portions of the spectrum. In other embodiments the emission devices may be arranged to emit wavelengths in the first or the second portions of the spectrum.

The electromagnetic emission device may be a mercury lamp such as a high pressure mercury lamp or a low pressure mercury lamp or a mercury free excimer lamp. The electromagnetic emission device may be a Xenon flash light. In other embodiments the electromagnetic emission device may be a light emitting diode (LED). The LED may comprise a semiconductor material in a solid state device. The chemistry of the semi-conductor material may be adjusted to control an emitted wavelength. Other UV emitting devices may be employed. Desirably the emitted wavelength is in an effective germicidal wavelength. The emitted wavelengths may be in the range from 100 nm to 280 nm. The device may comprise a semiconductor material arranged to produce light in a solid-state device. The wavelength emitted may be controlled or be tuneable by adjusting the chemistry of the semiconductor material.

In some embodiments the or each electromagnetic emission device may be a combination of discharge lamps and LED UV emitting devices. In some embodiments there are provided a plurality of electromagnetic emission devices. The plurality of devices may comprise a number of discharge lamps and a number of LED UV emitting devices. The emission devices may be arranged to emit in the first and/or the second portion of the spectrum.

In some embodiments the system may be arranged to emit electromagnetic radiation in a third portion of the spectrum. The system may comprise at least a third emission device or the first or second emission devices may be arranged to emit electromagnetic radiation in the third portion of the spectrum.

In some embodiments the electromagnetic emission device may be in the infrared range. It has been found that electromagnetic radiation in the infra-red range, particularly the near infra-red range, is effective against pathogens.

In some embodiments the electromagnetic emission device may be in the visible light range. It has been found that electromagnetic radiation in the visible light range is effective against pathogens.

It will be appreciated that electromagnetic radiation in the range from 200 nm to 280 nm can contribute to the disinfection of air or surfaces. Emissions in this range may be used to inactivate airborne pathogens. Emissions in this range may be used to inactivate pathogens such as bacteria and viruses on surfaces. In some embodiments the disinfection system is arranged primarily to disinfect air in the vehicle. In other embodiments the disinfection system is arranged primarily to disinfect surfaces in the vehicle. In some embodiments the disinfection system is arranged to disinfect air and surfaces in the vehicle.

It has been found that emissions in the range 200-284 nm (far UVC) can be used to inactivate pathogens such as viral pathogens. It has been found that UV in the range 207 to 222 nm is effective in killing pathogens such as airborne influenza virus. It has also been found that UV light in this range has significant inactivation efficiency against human coronaviruses including SARS-CoV-2. Such emissions are effective against viruses and bacteria which are extremely small and so UVC light is able to penetrate and kill the pathogens. However, it has been found that far UVC light does not cause significant health issues to humans. It is suggested that the far UVC light has a penetration range in biological materials of less than a few micrometers. As such it is believed that the far UVC light is not able to reach living human cells in the skin or eyes and is absorbed in the skin.

In some embodiments the electromagnetic emission devices output visible light. The visible light can be particularly effective against pathogens such as fungal agents.

In some embodiments the electromagnetic emission devices output infra-red radiation. In some embodiments the infra-red radiation may be in the near infra-red radiation. Near infra-red radiation may be particularly effective against pathogens such as bacterial agents.

It has been found that emissions around 254 nm have a significant effect on pathogens but also cause damage to DNA in human subjects.

Preferably the or each electromagnetic emission device has an output wavelength and an output intensity. Desirably the or each output wavelength can be adjusted and tuned to control the output wavelength.

In some embodiments the output wavelength may be variable. In some embodiments there may be a change from a first wavelength to a second wavelength wherein the first wavelength is different from the second wavelength.

When there is a change in wavelength from the first wavelength to the second wavelength there is a transition in electromagnetic radiation emitted from the first wavelength to the second wavelength. Preferably the controller outputs control signals specifying the transition from the first wavelength to the second wavelength. The transition may be a step transition or a linear transition or a parabolic transition. In some cases it may be desirable to provide a parabolic transition from the first wavelength to the second wavelength. Such a transition may be provided in varying a light output from the electromagnetic emission device.

In some embodiments the variation in the wavelength may be variable from a first wavelength to a second wavelength. In some embodiments an intensity of the radiation may be varied.

In at least some embodiments the output intensity can be controlled. The intensity may be varied in response to control signals output from the controller.

The output intensity may be reduced and then increased or vice versa. In some embodiments the output wavelength may be pulsed. The pulse may be a step change or may be a linear change or may be a parabolic change.

In some embodiments a duration of the output radiation may be controlled.

In some embodiments a dosage may be specified. The dosage may be controlled by controlling the output intensity and the duration of the electromagnetic radiation. The scenario selected by the controller may specify a high intensity emission for a short duration. Alternatively, the selected scenario may specify a lower intensity emission for a longer duration. It will be appreciated that the dosage is a function of the duration and the intensity of an emission.

It will be appreciated that the vehicle provides the power supply for the system. An advantage is that no ground power supply or support equipment is required to operate the disinfection system.

It will be appreciated that the autonomous nature of the vehicle and the disinfection system allows the vehicle to be disinfected without the system needing ground support. It will be appreciated that it may be desirable to disinfect the vehicle autonomously and without the need for external support.

In some embodiments the UV emission devices are arranged to operate to such that at least a portion of the vehicle is continuously disinfected. The UV emission devices may be arranged to operate continuously, that is at all times that the vehicle is in operation.

In other embodiments the UV emission devices may be arranged to operate in an ON/OFF cycle that effectively provides continuous disinfection. During an ON/OFF cycle there is a first period during which the at least one electromagnetic emission device is operating and a second period during which the at least one electromagnetic emission device is not emitting electromagnetic radiation. In some embodiments the intensity of output of the electromagnetic radiation is varied from a first value to a second value. The first value may be zero or substantially zero.

In some embodiments the disinfection system may further comprise visible light emission devices.

In some preferred embodiments the disinfection system may comprise at least a first electromagnetic emission device and a second electromagnetic emission device. The first and the second electromagnetic emission devices are adapted to emit radiation at different wavelengths. The first electromagnetic emission device may be different from the second electromagnetic emission device. The system may comprise for example UV emitting devices and visible light emitting devices. In some embodiments the system may additionally comprise infra-red emitting devices.

As described above the scenario may be in the form of a data table comprising data relating to vehicle. The scenario may include data relating to the vehicle, the location of the vehicle, inputs relating to external conditions in the location; occupancy of the vehicle. The scenario data table may have data that can be updated. The data that can be updated may comprise data and or information relative to a newly identified threat. This may be particularly relevant to military vehicles in which the mission profile may be updated with information such as data relating to a biological threat, such as details of a bacteria or a virus. Inputs to the scenario may be from the vehicle or platform such as Combat Management System modes. Such input may adapt the light output and control signals to the vehicle conditions.

In some embodiments the vehicle output comprises an output from a Combat Management System. The output form the Combat Management System may be input to the mission profile in the scenario or directly to the scenario data table

Data relating to the bacterial threat may be input to a mission profile data base and/or to one or more scenarios.

In some embodiments threat data may be uploaded to and may update one or more scenarios stored in the controller. Updated scenarios may output modified signals controlling the output electromagnetic radiation such as selected wavelength, intensity, and duration. Such modifications may vary depending on the type of threat that has been detected or may be present.

The vehicle may be provided with an interface through which inputs can be made.

An interface may be provided to input data to the mission profile. The interface may be wireless such as IR or may be an alternative such as a discrete or data bus inputs for example RS485/CAN/Ethernet. In some embodiments a manual input may be provided. The manual input may be a boost input. In some embodiments the manual input may be a manual control.

The electromagnetic emission devices may be installed in an interior of the vehicle. The devices may preferably be installed throughout the interior of the vehicle. Preferably the devices are installed and arranged such that the output radiation can contact substantially all surfaces in the interior of the vehicle. In other embodiments the devices are arranged such that the radiation can contact substantially all upper surfaces in the interior. A series of electromagnetic emission devices may be arranged through an interior of the vehicle. It may be that all of the emission devices are controlled to emit the same electromagnetic emissions. In some embodiments the electromagnetic emission devices may be arranged to emit different wavelengths. In some embodiments the electromagnetic emission devices may be different types of electromagnetic emission devices.

At least some of the electromagnetic emission devices are arranged to disinfect airborne viruses. In some embodiments the electromagnetic emission devices are arranged to disinfect airborne pathogens. The electromagnetic emission devises may be arranged to operate in combination with air moving means.

The or each electromagnetic emission device may comprise a mixed spectrum light sources emitting radiation. In some embodiments the light source may be tuneable to emit a controlled and specified wavelengths or a range of wavelengths. In some embodiments the wavelength may be varied between UV light and IR wavelengths.

In some embodiments the system may be arranged to emit electromagnetic radiation at a plurality of wavelengths. The system may be arranged to emit UV radiation centred around 200-284 nm (far UVC) and also in the range 207 to 222 nm. In other embodiments the system may be arranged to emit infra-red electromagnetic radiation in combination with UV electromagnetic radiation. Other combinations of wavelengths may be utilised depending on the sensitivity of the threat or threats.

The system may be described as being multispectral.

In other embodiments the or each electromagnetic emission devices comprises multiple emission sources. The multiple emission sources may be individually filtered and embedded in a single output.

The or each electromagnetic emission device may further comprise an EMI shield. In some embodiments the EMI shield may comprise a metal shield. In some embodiments the emission device may be a mercury free excimer lamp and a metal shield. The provision of EMI shielding facilitates maintenance of the overall EMI profile or footprint of the vehicle. It will be appreciated that in some circumstances the EMI profile of a vehicle may be specified in accordance with a specified use of the vehicle.

In some embodiments the or each emission device may be provided with a means of thermal management. In preferred embodiments the thermal management is passive. In some embodiments the passive thermal management may be provided by the use of thermal conductive heat sinks such as high heat polyamide heatsinks.

The power supply for the electromagnetic emission system is provided in the vehicle. The power supply may be a DC power supply. In some cases, the vehicle power supply may be an AC power supply. The power supply may be provided with an EMI filter. The power supply may further comprise a power converter. The power converter may be arranged to provide a suitable power supply to the disinfection system. The power supply may further comprise an output current control feedback. In some embodiments there may be provided a means of actively monitoring input current. In some embodiments means may be provided for monitoring the vehicle input voltage.

The controller is arranged to output control signals to the electromagnetic emission devices. The controller may be arranged to output a control signal. The output control signals may be arranged to control at least one of emission intensity; duration of emission; wavelength emitted; transition shape.

The controller may be arranged to control a duration of the first period during which the at least one electromagnetic device is emitting electromagnetic radiation. The controller may be arranged to control the intensity of the electromagnetic radiation emitted. The intensity may be measured in Watts. It will be appreciated that a combination of the intensity and duration of the electromagnetic radiation emitted may be used to calculate an applied dosage of the electromagnetic radiation.

In some circumstances it may be desirable to control the electromagnetic radiation emitted to have a high intensity for a short period of time. Such a dosage may be more suited to a situation in which the vehicle is not occupied and enables the disinfection is carried out rapidly. In other situations, where the vehicle is occupied, as may be input from the mission profile and/or scenario to output control signals controlling emission device to output lower intensity radiation but the duration may be increased to compensate and to deliver the same overall dosage.

The controller may be provided with a memory means storing electromagnetic emission device parameters such as wavelengths emitted by the electromagnetic emission device. The memory means may also be arranged to store data relating to the intensity of an electromagnetic emission device.

The controller may be provided as an integral part of an electromagnetic emission device. The electromagnetic emission device may be a lighting device. In other embodiments the controller may be provided separately from the electromagnetic emission device.

Information relating to emission device parameters may be configured by means of the interface in the course of manufacture in an “as built” configuration.

It will be appreciated that the operating temperature may affect the performance of the electromagnetic emission device. It is also appreciated that the duration of the temperature has an effect on the performance of the electromagnetic emission device. The controller may receive inputs relating to the temperature of the electromagnetic emission device and the duration of a period during which the electromagnetic emission device is at a temperature.

The controller may further store data relating to performance characteristics of the EMD device and the effect of temperature and temperature duration and aging of the electromagnetic emission device. The controller may further store compensation data such that the control signals are output to compensate for aging and temperature on the electromagnetic emission device.

The or each electromagnetic emission device may further have a temperature sensor. The temperature sensor may output a signal if a temperature threshold is reached. Desirably the controller receives the signal from the or each temperature sensor and may be arranged to prevent the electromagnetic emission from operating in the event of the temperature threshold being exceeded. In other embodiments a signal from the temperature sensor may instigate a shutdown. The shutdown may be temporary.

The or each electromagnetic emission device may comprise memory means arranged to store data in the device. The stored data may comprise calibration data specific to the electromagnetic emission device. The stored data may comprise temperature data and temperature history data.

It has been found that most materials do not reflect UV radiation. Instead, substantially all UV radiation may be absorbed. Consequently, disinfection by UV electromagnetic radiation is typically limited to direct line from the source.

It has been found that providing reflective surfaces in a vehicle may enhance the effectiveness of the disinfection system.

In some embodiments at least a surface may comprise a material, or be treated with a material, or covered by a material arranged to reflect UV radiation.

In accordance with an aspect of the invention there may be provided a vehicle wherein at least a surface may comprise a material, or be treated with a material, or covered by a material arranged to reflect UV radiation.

In some embodiments a surface may be treated with a coating, paint or lamination arranged to reflect UV radiation.

In accordance with an aspect of the invention a coating, paint or lamination may be provided or applied to least a surface in a vehicle in order to provide effective reflection of UV electromagnetic radiation. In one embodiment surfaces arranged to reflect UV electromagnetic radiation may be provided within the vehicle. It has been found that provision of such surfaces improves the disinfection of surfaces and air within the vehicle. Use of reflective surfaces will enhance the effectiveness of the UV within the vehicle by many factors in accordance with Sumpner's principle of irradiance. Use of more reflective surfaces significantly increases the effectiveness or irradiation and in some examples this may be as high as 30 times.

In some embodiments it can be expected that in a small, enclosed space surface which are more than 60% UV reflective or more than 80% UV reflective or more than 85% UV reflective or preferably more than 90% UV reflective surfaces can enhance an effectiveness of the disinfection system by up to four times. Improvement of the effectiveness may reduce disinfection times and/or allow the use of lower power levels for disinfection of a vehicle.

The reflective value of a surface may be measured as either specular reflection from a reflective surface such as glass or polished metal. With such surfaces the reflectance is very low at all angles except at the appropriate reflected angle.

Other surfaces may be diffuse surfaces which reflect radiation at all angles equally or near equally. Such surfaces are said to be Lambertian. With a Lambertian surface an apparent brightness to an observer is the same regardless of the observer's angle of view. The reflectivity of a surface may be measured using a spectrometer and compared to a suitable standard material which may be provided with a spectrometer.

In some cases reflectivity may vary with the wavelength of the emitted radiation. Desirably the specified reflectivity is measured in the UV range, that is from 100-400 nm.

In some embodiments a coating, paint or lamination may be arranged to be wavelength selective in terms of reflectivity. In other embodiments the coating, paint or lamination may be arranged to have a conversion properties such that UV electromagnetic radiation is produced from visible light.

In some embodiments the coating, paint or lamination may be arranged to have antibacterial additives.

In some embodiments reflective coatings or surfaces may be provided in areas that require particular attention such that the surface is properly disinfected. An example of such an area may be under sink taps and or under toilet basins. Other areas may be selected for particular attention for disinfection.

In other embodiments UV reflective surface may be combined with other material properties. In some examples it is possible to reflect particular visible and non-visible wavelengths to enhance the activation of surface properties. In some embodiments materials of a surface may be activated by particular wavelengths of light. In some embodiments surfaces such as the anti-bacterial and up conversion treatments may be activated.

In some embodiments UV reflective properties may be selected such that lower UV power levels can be utilised so lowering a UV dosage to below mandated limits for occupied areas.

In some embodiments continuous air and surface disinfection may be utilized with a lower UV dosage. Use of UV reflective surfaces may be arranged to enhance non line of sight/shaded surface disinfection.

In some embodiments air disinfection may be an important in combating threats such as Covid Sars etc.

It will be appreciated that the described vehicle and system for disinfection provides significant advantages and overcomes problems that have arisen in the past.

Historically vehicles have had to be immobilized or taken out of action in the event that a biological threat is identified on or in the vehicle. The biological threat may be bacterial, fungal, parasitic or viral threats. It is envisaged that other threats could arise. If a vehicle has to be immobilized due to the presence of the biological threat this can have a significant cost. This may be very significant when the vehicle is for example a civil aircraft or military vehicles such as aircraft, armored vehicles or aircraft carriers. The cost may not just be monetary but may further impact availability of the vehicle, and also strategic availability and posture. For example, an outbreak of Covid-19 on an aircraft carrier resulted in the aircraft carrier having to be side-lined for a period of time, during which the aircraft carrier was not able to be ready for action or to carry out strategic actions.

In some embodiments the vehicle receives an input from the vehicle sensors.

Inputs from the vehicle sensors are typically input to the mission profile or scenario. In some cases the input data may be from a mission profile. The controller is arranged to input the data to the mission profile and receive the outputs from the mission profile. The controller is arranged to output signals to the system in the vehicle and system. The output signals typically comprise wavelength, intensity, duration and transition time values based on the scenario selected from the input data and mission profile.

The disinfection system may comprise a program. The program may be a master program. The master program may provide a clock function in the controller. The disinfection system may comprise an additional program. The additional program may be referred to in this description as a slow program. The slow program may be provided to trigger a start of a program. The slow program may be used to start a program run in which the controller is arranged to check for input data; input the data into the mission profile; receive the output data and to output signals to the system. The slow program may be arranged to run from the clock program. The program run may be referred to a Scene Run function. The Scene Run function may be arranged to run every 10 ms. In some embodiments the scene run may be a mission duration. The scene run may be a repeat. The scene run may be infinite. The controller may comprise a Coordinate Converter. The coordinate converter may be a software program arranged to convert the output from the mission profile to the output data required to drive the electromagnetic emission devices. The output data may be arranged to control wavelength, intensity duration and transition time values. An output drive may be provided to send the calculated wavelength, intensity and duration and communicates the values to the electromagnetic emission device.

According to a second aspect of the invention there is provided a disinfection system for a vehicle in accordance with claim 1 the disinfection system comprising a controller and at least one electromagnetic emission device;

each electromagnetic emission device being arranged to emit radiation having a specified wavelength, intensity, and duration or dosage;

the controller being arranged to output signals to control at least one of the wavelength, intensity or duration;

the controller being arranged to generate the output signals to the electromagnetic emission device in response to a scenario output from the controller.

In accordance with the invention there is provided a vehicle 2 having a disinfection system 4 arranged to combat biological agents. The vehicle 2 has a power source (not illustrated) arranged to power the vehicle and to provide power to the disinfection system. The disinfection system comprises a controller, having a memory means and a number of electromagnetic emission devices 6. Each electromagnetic emission device 6 is arranged to emit radiation having a specified wavelength, intensity, duration or dosage. The vehicle is arranged to output signals to the controller. The controller is arranged to receive the output from the vehicle. The controller has a processor means and a memory means. The controller has a number of scenarios stored in the memory. The controller further has output means arranged to output control signals to the or each electromagnetic emission device. An output drive is arranged to transmit the output control signals to a plurality of electromagnetic emission devices provided in the vehicle. The control signals are arranged to control an output from the electromagnetic emission devices.

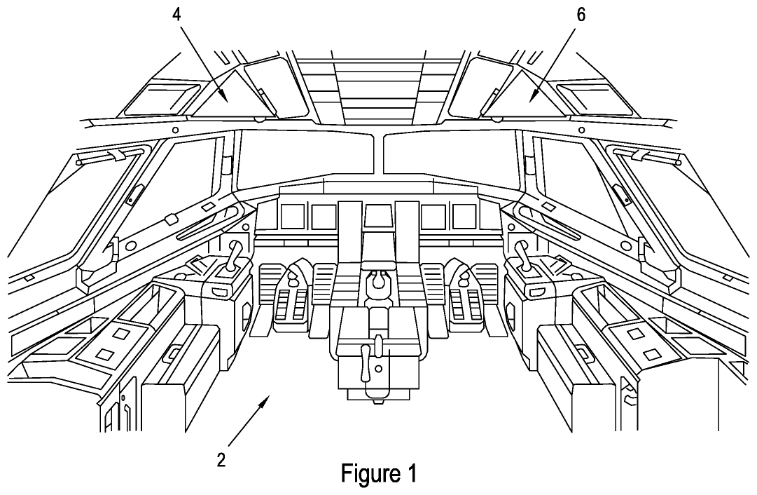

FIG. 1 schematically illustrates an aircraft in accordance with the invention. The aircraft has electromagnetic emission devices 6 located in the cabin.

Each electromagnetic emission device is arranged to emit electromagnetic radiation. The or each electromagnetic emission device outputs radiation having at least one of a specified intensity; a specified wavelength; a specified duration.

The output control signals are generated in response to the vehicle output. The vehicle output or outputs are indicative of a status of the vehicle. The status can be indicative of whether the vehicle is stationary, moving, occupied or not. The status can also indicate whether a door is open. The vehicle is also arranged to provide an output from a vehicle mission profile usage. In some embodiments the vehicle is arranged to output signals from a combat management system of the vehicle. The output from the vehicle may also be a manual input such as a boost input or a manual override. The vehicle is provided with a number of sensors (not illustrated). The sensors may be selected for example from one or more of door locked, proximity switches, status, movement detection sensors, aircraft weight on wheels sensors, position coordinates, combat management system data or status, internal temperature; ambient temperature, occupancy sensors. It will be appreciated that this is a limited selection of sensors and the skilled person will appreciate that sensors for other parameters may be provided.

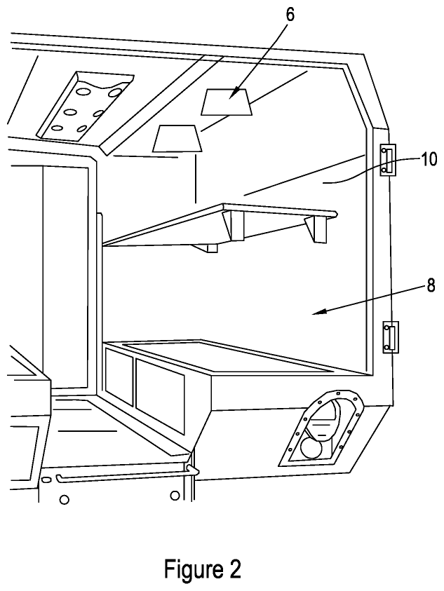

FIG. 2 schematically illustrates a ground vehicle 8 in accordance with the invention. The vehicle has electromagnetic emission devices 6 located in a rear portion 10 of the vehicle.

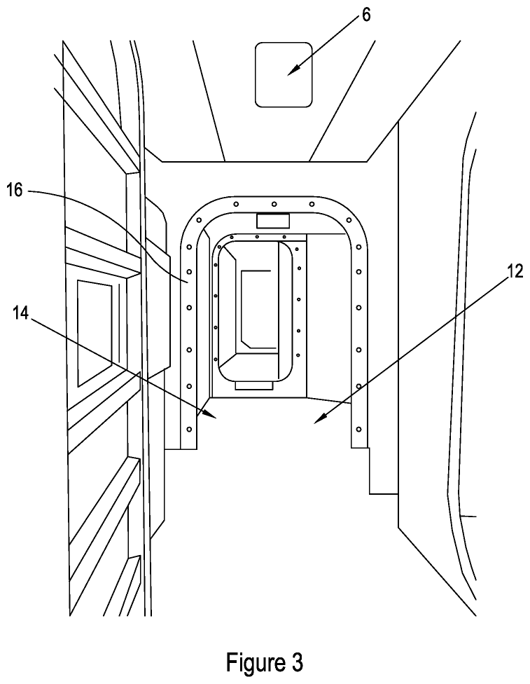

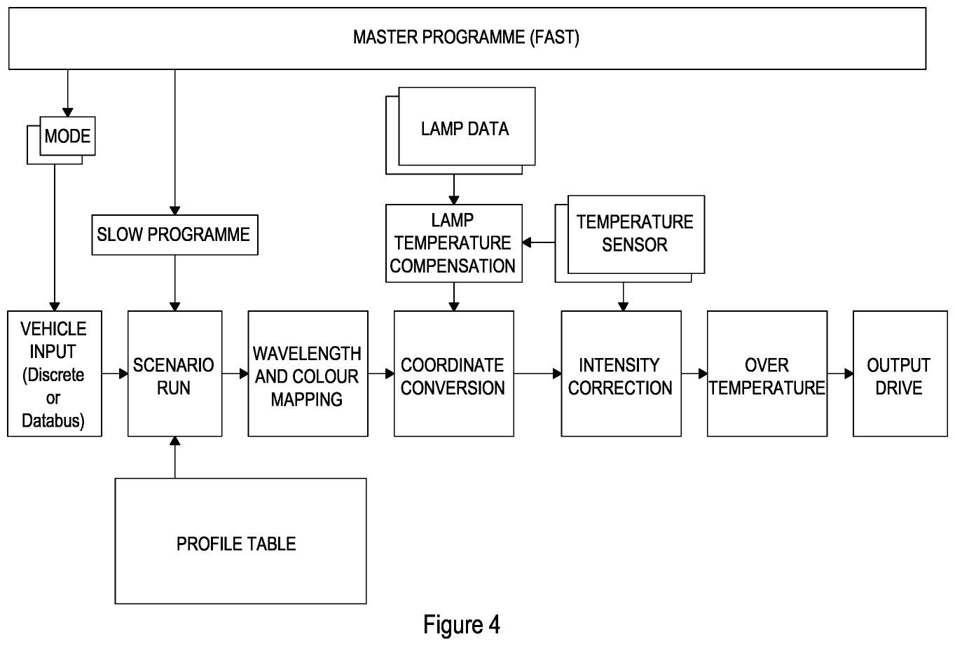

FIG. 3 schematically illustrates a ship in accordance with the invention. The ship 12 has electromagnetic emission devices 6 located for example in a corridor area 14 of the ship between bulkheads 16.

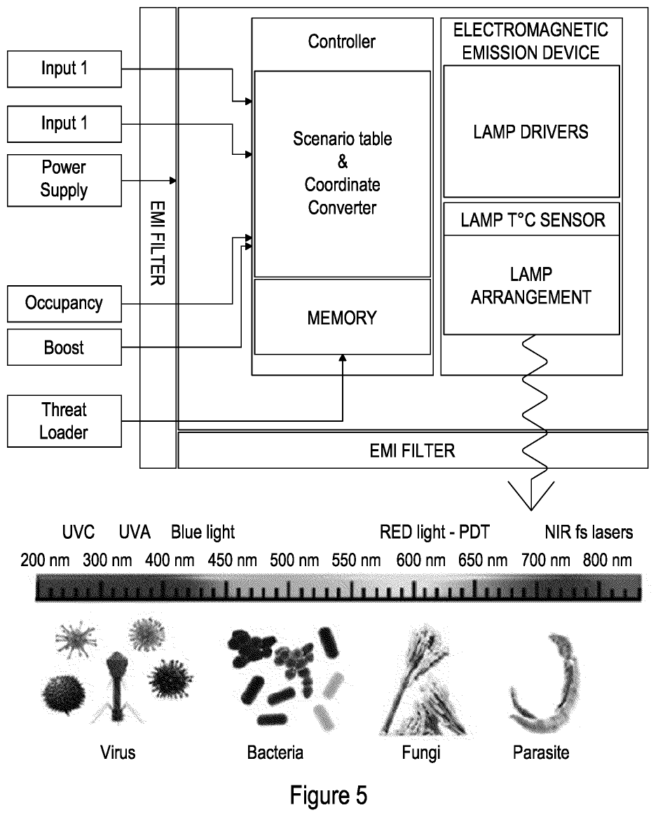

FIG. 5 is a schematic block diagram of the disinfection system 18. The controller 20 has a memory means 22. The controller has a number of scenarios stored in the memory means. The scenario can be a data table. In some cases the scenario output may be a mission profile data table. In some cases the controller may have a number of scenarios. The controller receives a number of inputs from the vehicle. For example the vehicle mission usage may output a signal that an aircraft is taxiing. The controller is arranged to use the inputs from the vehicle to select a scenario from the stored scenarios. For example a signal that the vehicle is taxiing will be part of the logic used to select an appropriate scenario. The scenario may also be selected in response to inputs from a sensor or a human input. The controller may be arranged to use a human or manual input or override other inputs in selecting a scenario.

The controller is arranged to output control signals to the electromagnetic emission device or devices by the output means. The output signals are received by the or each electromagnetic emission device. The or each electromagnetic emission device is provided with a lighting control profile which controls the radiation emitted by the electromagnetic emission device.

The lighting control profile can be set up to be compatible with the vehicle interior materials. A desired output from the or each electromagnetic emission device can be selected to take account of the arrangement of the interior surfaces, the materials used for the interior surfaces and airflow through the vehicle.

The or each electromagnetic emission device may have sensors in the device arranged to measure a temperature of the device or current applied or voltage. The electromagnetic emission device may be arranged to output feedback signals to the controller.

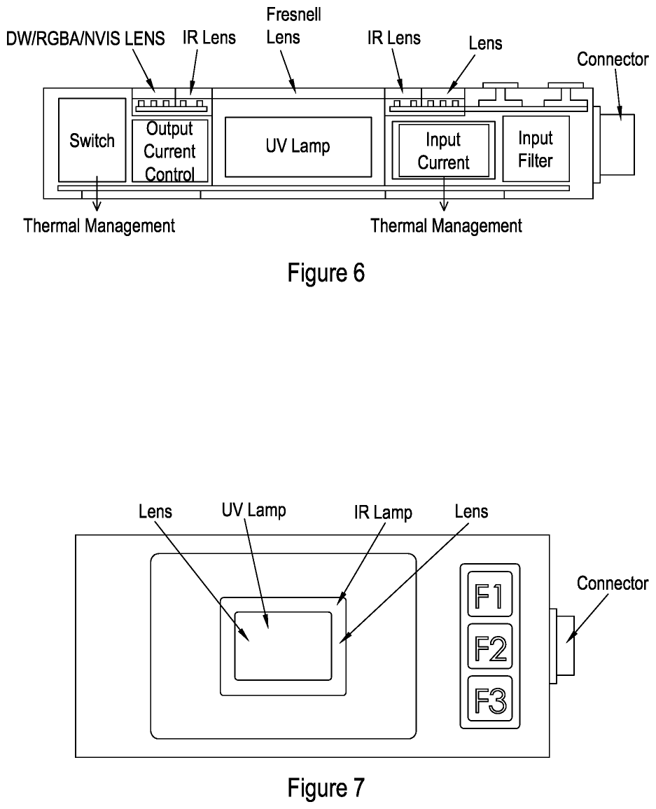

As described above and illustrated in FIG. 5 or 6 a vehicle input is received and is input to the controller in the disinfection system. In response to the input a scenario is selected and run. The scenario generates a desired wavelength, intensity, duration and desired dosage for the or each electromagnetic emission devices. The output signals from the controller are directed to a coordinate convertor. The coordinate convertor applies information from the aging and calibration data to calculate and apply an intensity correction. Further information may be applied from temperature sensors in the electromagnetic emission device, particularly electromagnetic emission device temperature. After application of information regarding the ageing and calibration data and temperature information signals are output to an output drive. The signals from the output drive are transmitted to the electromagnetic emission devices and electromagnetic radiation is emitted in accordance with the control signals and the device lighting control profile.

Each electromagnetic emission device is arranged to output electromagnetic emission radiation. A first electromagnetic emission device can have LED sources. The LED sources are arranged to emit electromagnetic radiation in the UV range. The LED sources are tuneable and the wavelength can be selected to be effective to inactivate any airborne or surface pathogens. In one embodiment the LED sources are tuneable to output UV light. An output may in some examples be from 250 nm to 260 nm or for example between 253 nm and 255 nm. Emissions around 254 nm have been found to be particularly effective against pathogens such as viruses and SARS-CoV-2 virus.

It will be appreciated that other electromagnetic wavelengths may be found to be more effective against other pathogens such as bacterial pathogens, fungal pathogens, or parasites. For example it has been found that visible light is effective against fungal pathogens. It has also been found that near infra-red radiation is effective against at least some bacterial pathogens.

The electromagnetic radiation can be arranged to have a variable intensity. In other embodiments the electromagnetic radiation is arranged to have a variable dosage. The dosage is controlled by selecting a desired intensity and duration.

In some arrangements the or each electromagnetic emission device may be a mercury free excimer lamp. Some electromagnetic emission devices are provided with EMI shields. An EMI shield may be desirable to ensure that an EMI signature of the vehicle is not changed by operation of the disinfection system.

Each electromagnetic emission device may be provided with a passive heat management system in the form of a heatsink.

Each electromagnetic emission device can be provided with a temperature sensor arranged to sense a temperature of the electromagnetic emission device.

In some cases an ambient temperature sensor may be provided in the vehicle and ambient temperature data input to the controller. In some cases an external ambient temperature sensor may be provided and external temperature data input to the controller.

The system can be programmed to output UV light or other electromagnetic radiation at an intensity, duration and dosage that is effective to inactivate pathogens. In some embodiments a wavelength of the electromagnetic radiation emitted by the electromagnetic emission device may be varied. In other embodiments the intensity of the electromagnetic emission radiation emitted may be varied. In some embodiments the disinfection system is arranged to vary both wavelength and intensity of the radiation emitted. The variation may be sequential or simultaneously.

Input sensors may indicate that the vehicle is not occupied; such as an aircraft during change over. The scenario for an unoccupied vehicle may output higher intensity but shorted duration emissions. It will be appreciated that the vehicle can be disinfected more rapidly. Another scenario for an unoccupied vehicle may output signals such that the electromagnetic emission devices are arranged to output emissions at 254 nm which is effective against pathogens but cannot be used when the vehicle is occupied as emissions at 254 nm are harmful to humans.

It will be appreciated that the emissions output from the electromagnetic emission devices may be selected to be effective against a pathogen.

In some embodiments an input may be input to the controller relating to a specific pathogen and effective wavelengths for inactivation the specific pathogen.

The electromagnetic emission devices are provided throughout the vehicle. A plurality of electromagnetic emission devices are provided throughout the vehicle. The electromagnetic emission devices may all be the same electromagnetic emission device. Alternatively there may be more than one form of electromagnetic emission device installed in the vehicle.

The controller is arranged to store calibration data relating to the electromagnetic emission devices installed in the vehicle.

The controller may also be arranged to store information relating to aging of the electromagnetic emission devices or the effect of temperature on the electromagnetic emission devices.

The or each scenario is stored in the controller.

The disinfection system is provided with a master program which inputs a time to the controller. The master program provides a clock for the system to run scenario timing.

The disinfection system is also provided with a “slow program” which is arranged to trigger a scenario run. The slow program may be arranged to trigger a scenario run every 10 ms.

The scenario run directs the controller to take input data from the vehicle and to run the scenario and to output signals to the output driver and to the control the electromagnetic emission devices in accordance with the selected scenario.

The power for the disinfection system is provided in the vehicle.

An example of an electromagnetic emission device is illustrated in FIG. 6. In this illustrated embodiment the controller is provided integral with the electromagnetic emission device. The electromagnetic emission device comprises a UV radiation device and an IR radiation device. The controller is arranged to receive output signals from the vehicle. The vehicle output signals are processed by the controller to select a scenario from the scenarios stored in the memory. The controller is arranged to output signals to the electromagnetic emission device. The electromagnetic emission device receives the controller output signals. The electromagnetic emission device comprises a lighting control profile. The controller output signals control an electromagnetic output from the device in accordance with the lighting control profile stored in the electromagnetic emission device.

The electromagnetic emission device is provided with thermal management system in the form of a heatsink. The output from the electromagnetic emission device passes through a respective lens. The controller is provided within the device in this embodiment.

The vehicle and disinfection system are arranged to be independent of ground support. In some vehicles, such as private jets, ground support is not available. It is desirable therefore that the disinfection system is operable independently. It is also desirable that the disinfection system can be used to be operative against a variety of pathogens and an input to the controller may select an alternative electromagnetic emission device in order to tune the disinfection system to be effective against a pathogen of concern.

What is claimed is:

1. A vehicle having a disinfection system arranged to combat biological, viral, fungal and parasitic agents,
the vehicle having a power source;
the vehicle having an operational status;
the vehicle being arranged to generate at least one vehicle output indicative of the vehicle operational status; and
the disinfection system comprising:
at least one electromagnetic emission device arranged to emit electromagnetic radiation having at least one of:
a specified intensity;
a specified wavelength;
a specified duration;
a controller having a processor means and a memory means;
a plurality of scenarios arranged to be stored in the memory means;
the controller being arranged to receive a signal indicative of operational status;
wherein the controller is arranged to use the signal indicative of the operational status to select a scenario from the plurality of scenarios stored in the memory means;
wherein the controller has output means arranged to output first output control signals in response to the selected scenario to the at least one electromagnetic emission device to control at least one of the intensity, wavelength, or duration of the emitted electromagnetic radiation; and
wherein the at least one electromagnetic emission device emits radiation in response to the first output control signals.

2. The vehicle of claim 1, wherein the or each scenario comprises a data table stored in the memory means in the controller.

3. The vehicle of claim 1, wherein the scenario is selected in response to vehicle output from a sensor or a human input.

4. The vehicle of claim 1, wherein the controller is a human or manual input is arranged to override other inputs in selecting a scenario.

5. The vehicle of claim 1, wherein the vehicle output comprises at least one of a vehicle mission profile or a manual input.

6. The vehicle of claim 1, wherein the disinfection system comprises a plurality of electromagnetic emission devices arranged to emit electromagnetic radiation a plurality of the electromagnetic emission devices emit radiation at the same wavelength.

7. The vehicle of claim 6, wherein the disinfection system comprises a plurality of electromagnetic emission devices and wherein at least one emission device emits electromagnetic radiation in a first portion of the spectrum and at least one further emission device is arranged to emit electromagnetic radiation in a second portion of the spectrum.

8. The vehicle of claim 1, wherein the disinfection system comprises at least one electromagnetic emission device emitting electromagnetic radiation at least at a first wavelength and a second wavelength wherein the first wavelength is different from the second wavelength.

9. The vehicle of claim 8, wherein the controller outputs signals to the or each electromagnetic device to control a transition of the electromagnetic radiation from the first wavelength to the second wavelength.

10. The vehicle of claim 1, wherein an intensity of the electromagnetic radiation emitted by at least one electromagnetic device is variable.

11. The vehicle of claim 1, wherein a duration and intensity of the electromagnetic radiation emitted by at least one electromagnetic device is controlled by the controller in accordance with a scenario selected by the controller.

12. The vehicle of claim 1, wherein the emitted electromagnetic radiation is in the range from 1 nm to 2000 nm.

13. The vehicle of claim 1, wherein at least one electromagnetic emission device comprises a lighting control profile stored in the electromagnetic emission device.

14. The vehicle of claim 1, wherein the or each electromagnetic emission device is selected from the group consisting of a LED unit, a low pressure mercury lamp, and a mercury free excimer lamp.

15. The vehicle of claim 1, wherein the at least one electromagnetic emission device is arranged to emit light in the near infra-red range.

16. The vehicle of claim 1, wherein the or each electromagnetic emission device comprises an electromagnetic shield.

17. The vehicle of claim 1, wherein the vehicle further comprises at least one of the following: a surface of a material; a surface treated with a material; a surface covered by a material wherein the material reflects at least 80% UV radiation.

18. A disinfection system for a vehicle, the disinfection system comprising a controller having a memory means and at least one electromagnetic emission device;
the or each electromagnetic emission device being arranged to emit radiation having a specified characteristic, the specified characteristic being selected from the group consisting of wavelength, intensity, and duration;
the controller being arranged receive signals from a vehicle in which the system is installed in use, the signals being indicative of an operational status of the vehicle;
the controller being arranged to select a scenario from a plurality of scenarios stored in the memory means in response to the signal indicative of the operational status and the controller being arranged to output signals to control the specified characteristic emitted by the or each electromagnetic emission device in response to the selected scenario.

19. A vehicle having at least one surface to reflect at least 80% UV radiation.

20. The vehicle of claim 19, wherein the surface is selected from the group consisting of:
a surface comprising a material arranged to reflect at least 80% UV radiation;
a surface treated with a material arranged to reflect at least 80% UV radiation; and
a surface covered by a material arranged to reflect at least 80% UV radiation.
